(19) United States
(12) Patent Application Publication
(10) Pub. No.: US 2008/0306261 A1
(43) Pub. Date: Dec. 11, 2008

(54) Process For Treating a Reaction Mixture Comprising Alkoxycarbonylaminotriazine

(75) Inventors: Jörg Schneider, Wezembeek-Oppern (BE); Gunter Scherr, Ludwigshafen (DE); Rainer Erhardt, Mannheim (DE); Andreas Eichfelder, Maxdorf (DE); Martin Reif, Romerberg (DE); Stefan Hirsch, Neustadt an der Weinstrass (DE); Georg Sieder, Bad Durkheim (DE); Thomas Holtmann, Speyer (DE); Jurgen Ciprian, Ludwigshafen (DE); Hermann Ascherl, Dirmstein (DE)

Correspondence Address: CONNOLLY BOVE LODGE & HUTZ, LLP, P O BOX 2207, WILMINGTON, DE, 19899, US

(73) Assignee: BASF Aktiengesellschaft, Lidwigshafen (DE)

(21) Appl. No.: 11/916,626

(22) PCT Filed: Jun. 2, 2006
(86) PCT No.: PCT/EP2006/062861

§ 371 Date: Dec. 5, 2007

(30) Foreign Application Priority Data
Jun. 6, 2005 (DE) 10 2005 025 899.9

(51) Int. Cl. C07D 251/48

(52) U.S. Cl. 544/204

(57) ABSTRACT

Processes comprising: providing an alkanolic reaction mixture, the alkanolic reaction mixture comprising: at least one alkoxycarbonylaminotriazine; at least one carbonic ester; at least one C₁-C₁₃-alkanol, which alkanol may further comprise up to two ether-bound oxygen atoms and may further comprise a substituent selected from the group consisting of C₁-C₄-alkyls and hydroxyls; and at least one alkali metal alkoxide or alkaline earth metal alkoxide; and subjecting the alkanolic reaction mixture to extraction with a polar extractant such that at least a portion of polar/ionic components present in the reaction mixture are removed, wherein the polar extractant is less than entirely miscible with an organic phase of the reaction mixture, to form an alkanolic phase comprising the at least one alkoxycarbonylaminotriazine and a polar phase comprising the polar extractant and the portion of polar/ionic components; and wherein the extraction is carried out with one or more selected from the group consisting of mixer/settler units, columns, centrifugal field separation extractors, and combinations thereof.

The invention relates to a process for working up an alkanolic reaction mixture which is obtained in the preparation of alkoxycarbonylaminotriazines and comprises at least one alkoxycarbonylaminotriazine, at least one cyclic and/or acyclic carbonic ester, at least one C₁-C₁₃-alkanol which optionally comprises one or two oxygen atoms as an ether bond and is optionally substituted by C₁-C₄-alkyl and/or hydroxyl, and also at least one alkali metal or alkaline earth metal alkoxide, with or without melamine and with or without catalyst.

The preparation of alkoxycarbonylaminotriazines by reacting triazines, for example melamine, with carbonic esters in the presence of a base is known, for example, from EP-A 0 624 577. In this preparation, melamine is generally reacted with a carbonic ester in the presence of the parent alkanol of the carbonic ester and in the presence of an alkali metal alkoxide based on the parent alcohol of the carbonic ester as a base. For workup, a mineral acid is added to the reaction mixture for neutralization. Suitable acids mentioned are phosphoric acid, sulfuric acid and/or hydrochloric acid. The alkoxycarbonylaminotriazine is subsequently obtained by an extraction with an organic solvent and the evaporation of the solvent. Alternatively, after the addition of the acid, a solid is isolated by filtration and is then washed and dried.

WO-A 03/035628 discloses a process for preparing alkoxycarbonylaminotriazines, in which the reaction mixture is worked up by first neutralizing with a preferably aqueous acid. Suitable acids mentioned are nitric acid, sulfuric acid, phosphoric acid or mixtures thereof but also formic acid. After the addition of the acid to the reaction mixture, an aqueous and an alkanolic phase are formed and are separated from one another. The alkanolic phase comprises the alkoxycarbonylaminotriazine. To increase the concentration of alkoxycarbonylaminotriazine, the organic phase is concentrated after the removal of the aqueous phase.

A corresponding process for working up a reaction mixture comprising alkoxycarbonylaminotriazine is also disclosed in WO-A 2004/054990.

WO-A 2004/041922 discloses a preparation and workup process for carbamate-melamine-formaldehyde crosslinkers. In this process, the workup is likewise effected by addition of an acid, for example sulfuric acid, formic acid, oxalic acid, phosphoric acid, hydrochloric acid or mixtures thereof. The salt formed in the neutralization is removed by filtration and washing with water.

It is an object of the present invention to provide a process for working up an alkanolic reaction mixture comprising at least one alkoxycarbonylaminotriazine, which allows removal of salts and any further polar components from a reaction mixture comprising at least one alkoxycarbonylaminotriazine, and also the controlled influencing of a plurality of components of different polarity in the reaction mixture without great apparatus complexity.

The object is achieved by a process for working up an alkanolic reaction mixture which is obtained in the preparation of alkoxycarbonylaminotriazines and comprises at least one alkoxycarbonylaminotriazine, at least one cyclic and/or acyclic carbonic ester, at least one C₁-C₁₃-alkanol which optionally comprises one or two oxygen atoms as an ether bond and is optionally substituted by C₁-C₄-alkyl and/or hydroxyl, and also at least one alkali metal alkoxide or alkaline earth metal alkoxide, with or without melamine and with or without catalyst, in which polar and/or ionic components are removed by extraction with a polar extractant which is not entirely miscible with the organic phase present in the reaction mixture, to obtain an alkanolic phase comprising at least one alkoxycarbonylaminotriazine and a polar phase comprising extractant with polar and/or ionic components dissolved therein.

Preferred alkoxycarbonylaminotriazines are those of the general formula (I)

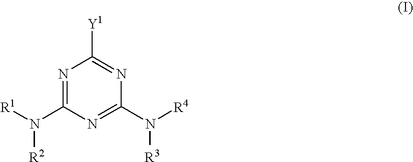

in which the symbols and indices are each defined as follows:

Y¹ is hydrogen, C₁-C₄-alkyl, phenyl optionally substituted by C₁-C₄-alkyl, C₁-C₄-alkoxy, or halogen, or a radical of the formula NR⁵R⁶ and

R¹, R², R³, R⁴, R⁵ and R⁶ are each independently hydrogen or a radical of the formula COOX or X, or selected from the group of (—CH₂—O)₁—H, (—CH₂—O)₁—R, (—CH₂—O)ₖ—CH₂—N(Z)-Q and (—CH₂—O)ₖ—CH₂—N(Z)-Q, where
k is from 0 to 10, preferably from 1 to 5, more preferably 1 or 2 and in particular 1, and 1 is from 1 to 10, preferably from 1 to 5, more preferably 1 or 2 and in particular 1,
R is selected from the group of alkyl, cycloalkyl and alkylaryl, where the R groups comprise preferably fewer than 13 carbon atoms and R is preferably a C₁-C₁₃-alkyl and more preferably methyl or butyl,
Q is a triazine radical of the general formula (II)

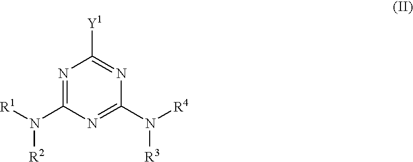

X is C₁-C₁₃-alkyl whose carbon skeleton may be interrupted by 1 or 2 nonadjacent oxygen atoms in ether function and/or substituted by hydroxyl, or is C₃-C₆-alkenyl and
Z is an R¹, R², R³, R⁴, R⁵ or R⁶ radical as defined above,
   and
   at least one of the R¹ to R⁴ radicals or, when Y¹ is NR⁵R⁶, at least one of the R¹ to R⁶ radicals, is COOX.

C₁-C₄-Alkyl is, for example, methyl, ethyl, propyl, isopropyl, butyl sec-butyl or tert-butyl.

Phenyl optionally substituted by C₁-C₄-alkyl, C₁-C₄-alkoxy or halogen is, for example, phenyl, 2-, 3- or 4-methylphenyl, 2-, 3- or 4-ethylphenyl, 2,4-dimethylphenyl, 2-, 3- or 4-methoxyphenyl, 2-, 3- or 4-ethoxyphenyl, 2,4-dimethoxyphenyl, 2-, 3- or 4-fluorophenyl or 2-, 3- or 4-chlorophenyl.

C₁-C₁₃-Alkyl whose carbon skeleton may be interrupted by 1 or 2 nonadjacent oxygen atoms in ether function and/or substituted by hydroxyl is, for example, pentyl, isopentyl, neopentyl, tert-pentyl, hexyl, 2-methylpentyl, heptyl, octyl, 2-ethylhexyl, isooctyl, nonyl, isononyl, decyl, isodecyl, undecyl, dodecyl, tridecyl, isotridecyl, 2-methoxyethyl, 2-ethoxyethyl, 2-propoxyethyl, 2-butoxyethyl, 2- or 3-methoxypropyl, 2- or 3-ethoxypropyl, 2- or 3-propoxypropyl, 2- or 4-methoxybutyl, 2- or 4-ethoxybutyl, 3,6-dioxaheptyl, 3,6-dioxaoctyl, 3,7-dioxaoctyl, 4,7-dioxaoctyl, 2- or 3-butoxypropyl, 2- or 4-butoxybutyl, 2-hydroxyethyl, 2- or 3-hydroxypropyl, 2- or 4-hydroxybutyl, 3-hydroxybut-2-yl. (The above-named isooctyl, isononyl, isodecyl and isotridecyl are trivial names and stem from the alcohols obtained by the oxo process—cf Ullmann's Encyclopedia of Industrial Chemistry, 5th Edition, Vol. A1, pages 290 to 293, and also Vol. A10, pages 284 and 285).

C₃-C₆-Alkenyl is, for example, allyl, methallyl, ethallyl, 2-, 3- or 4-penten-1-yl or 2-, 3-, 4- or 5-hexen-1-yl.

C₁-C₁₃-Alkanol which optionally comprises one or two nonadjacent oxygen atoms as an ether bond and is optionally substituted by C₁-C₄-alkyl and/or hydroxyl is, for example, methanol, ethanol, propanol, isopropanol, butanol, isobutanol, sec-butanol, tert-butanol, pentanol, isopentanol, neopentanol, tert-pentanol, hexanol, 2-methylpentanol, heptanol, octanol, 2-ethylhexanol, isooctanol, nonanol, isononanol, decanol, isodecanol, undecanol, dodecanol, tridecanol, isotridecanol, 2-methoxyethanol, 2-ethoxyethanol, 2-propoxyethanol, 2-butoxyethanol, 2- or 3-methoxypropanol, 2- or 3-ethoxypropanol, 2- or 3-propoxypropanol, 2- or 4-metboxybutanol, 2- or 4-ethoxybutanol, 3,6-dioxaheptanol, 3,6-dioxaoctanol, 3,7-dioxaoctanol, 4,7-dioxaoctanol, 2- or 3-butoxypropanol, 2- or 4-butoxybutanol, ethane-1,2-diol, propane-1,2-diol, propane-1,3-diol, 3-oxa-5-hydroxypentanol, 3,6-dioxa-8-hydroxyoctanol, 3-oxa-5-hydroxy-2,5-dimethylpentanol or 3,6-dioxa-8-hydroxy-2,5,8-trimethyloctanol.

The C₁-C₁₃-alkanol which optionally comprises one or two nonadjacent oxygen atoms as an ether bond and is optionally substituted by at least one C₁-C₄-alkyl- and/or hydroxyl is more preferably selected from methanol, ethanol, propanol, isopropanol, butanol, isobutanol, sec-butanol, tert-butanol, pentanol, isopentanol, neopentanol, tert-pentanol, hexanol, 2-methylpentanol and heptanol or mixtures thereof.

Very particular preference is given to butanol, isobutanol, sec-butanol and tert-butanol and to mixtures of methanol and butanol.

A cyclic carbonic ester is a carbonate of the general formula (III)

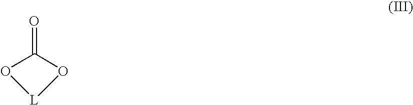

in which

L is ethylene, 1,2- or 1,3-propylene or 1,2-, 1,4-, 2,3- or 1,3-butylene.

Acyclic carbonic esters are, for example, diaryl carbonate, dialkyl carbonate, aryl alkyl carbonate and dialkenyl carbonate. The acyclic carbonic ester is preferably selected from carbonates of the general formula (IV)

Z¹O—CO—OZ² (IV)

in which

Z¹ and Z² are each independently alkyl, cycloalkyl and aryl. The Z¹ and Z² radicals preferably comprise fewer than 13 carbon atoms. More preferably, Z¹ and z² are a C₁-C₈-alkyl and in particular methyl or butyl.

Preferred dialkyl carbonates are dimethyl carbonate, diethyl carbonate, dipropyl carbonate, dibutyl carbonate and methyl butyl carbonate.

Preferred aryl alkyl carbonates are methyl phenyl carbonate or butyl phenyl carbonate.

Suitable diaryl carbonates are, for example, diphenyl carbonate, di(para-tolyl) carbonate, di(α-naphthyl) carbonate or di(β-naphthyl) carbonate.

A preferred dialkenyl carbonate is diallyl carbonate.

Particularly preferred carbonic esters are dimethyl carbonate, diethyl carbonate, dibutyl carbonate, methyl butyl carbonate, diphenyl carbonate, propylene carbonate or mixtures thereof.

Suitable alkali metal or alkaline earth metal alkoxides are, for example, lithium, sodium, potassium, magnesium or calcium salts of the alkanols designated in detail above. The use of alkali metal methoxides, especially of sodium methoxide, is preferred. The alkali metal or alkaline earth metal alkoxide can be used either in the solid state or in dissolved or suspended form.

Preferred solvents/diluents are in this case especially the alcohols designated in detail above, alone or as a mixture with one another. However, other customary inert diluents known per se may also be used.

Catalysts which may be present in the reaction mixture are catalysts which are used to prepare the alkoxycarbonylaminotriazine. Such catalysts are, for example, phase transfer catalysts, as described in Ullmann's Encyclopedia of Industrial Chemistry, 5th Edition, Vol. A19, pages 239 to 248. Further catalysts may be metal salts or complexes, preferably oxides, chalcogenates, carbonates or halides of the alkali metals, alkaline earth metals or transition metals. Mention should be made here in particular of lithium chloride, magnesium chloride or sodium carbonate.

Ionic components which are present in the reaction mixture are generally dissociated salts. According to the invention, they are removed by extraction with a polar extractant which is not entirely miscible with the organic phase present in the reaction mixture.

Furthermore, polar components may, in accordance with the invention, be removed from the reaction mixture by the extraction with the polar extractant which is not entirely miscible with the organic phase present in the reaction mixture.

Salts in the reaction mixture may form, for example, when acid is added to the alkanolic reaction mixture for neutralization or when the alkanolic reaction mixture is introduced into an acid. The acid may be either in concentrated form or diluted with water. A uniform distribution of the acid in the reaction mixture is achieved by ensuring suitable mixing during the metered addition of the acid.

For neutralization of the reaction mixture, it is possible to use all customary and industrially available organic and inorganic acids in any concentration, preferably as 30-85% by weight aqueous solutions. Preference is given to using mineral acids whose salts have a high water solubility, such as nitric acid, sulfuric acid or phosphoric acid. A further suitable acid is formic acid. According to the invention, particular preference is given to the use of nitric acid.

The extraction is carried out with a polar extractant which is not entirely miscible with the organic phase to obtain an alkanolic phase comprising alkoxycarbonyl-aminotriazine and a polar phase which comprises extractant with ionic and/or polar components dissolved therein. In this context, not entirely miscible means that two phases with different composition form, and not entirely miscible is also understood to mean that the extractant and the organic phase do not mix at all. A preferred extractant is water, and particular preference is given to fully demineralized water.

In addition to the polar extractant, phase separation assistants may be added in the extraction, as are described, for example, in Ullmann's Encyclopedia of Industrial Chemistry, Sixth Edition, 2000 Electronic Release, Section: Emulsion, Chapter 6, Breaking of Emulsions. Phase separation assistants are, for example, organic solvents, substances with surfactant character such as phenolsulfonic acid condensates, polyimines or polyacrylates, emulsion breakers and demulsifiers. Salts may also be used in certain cases. Phase separation assistants suitable in accordance with the invention are, for example, sepabase® from Baker Petrolite, Sugar Land, Tex. or Sokalan® CP, Sokalan® CP5, Sokalan® CP9 and Sokalan® HP25 from BASE AG, Ludwigshafen. However, preference is given to not using any phase separation assistants.

When a neutralization of the alkanolic reaction mixture is carried out this and the removal by extraction of the salts formed by the neutralization can be effected in one step or in separate process steps. Preference is given to effecting the neutralization and the removal of the salts formed by the neutralization in two steps.

For the extraction, the apparatus known to those skilled in the art, for example mixer/settler units, columns with or without energy input or extractors which are based on the principle of centrifugal field separation, may be used. A mixer/settler unit comprises generally a mixer unit such as a stirred vessel, a mixing pump, a nozzle or a static/dynamic mixer. The mixer/settler unit further comprises a separator which is generally designed as a horizontal vessel with or without internals.

Suitable columns which can be used for the extraction are, for example, columns with structured packing or random packing, or sieve tray columns. Suitable sieve tray columns are, for example, also crossflow sieve tray columns. The random packings used may be all random packings known to those skilled in the art. Such random packings are described, for example, in Klaus Sattler, Thermische Trennverfahren [Thermal separating processes], 2nd Edition, VCH Verlagsgesellschaft mbH, Weinheim, 1995, pages 226 to 229.

Suitable structured packings are ordered or unordered packings. Such structured packings are, for example, lamella packings, fabrics, drawn-loop or formed-loop knits.

The random packings, structured packings or sieve trays may be manufactured from metal or plastic. Owing to the good wetting properties of metals, preference is given to using metal as the material for the random packings, structured packings or sieve trays when the wash phase is selected as the continuous phase. Particularly suitable metals are stainless steels.

In addition to the operation of the columns with random packings, structured packings or trays with and without pulsation, it is also conceivable to use them without internals, for example as a spray column. Examples of suitable commercial extraction columns with mechanical stirrer systems are rotary disk extractors, Old-Rushton columns, Kühni extractors, stirred cell extractors, Graesser extractors. However, it is also possible to use centrifugal extractors such as Podbielniak extractors or Lurgi-Westfalia extractors.

In the extraction, the wash phase comprising the polar extractant which is not entirely miscible with the organic phase may form either the continuous or the disperse phase of the extraction. In a preferred form of the extraction, the wash phase forms the continuous phase.

In addition to the use of a single extraction apparatus, it is also possible to carry out the extraction in a plurality of apparatuses. In this case, a combination of different apparatus types may also be used. A preferred combination is formed by a mixer/settler unit and a column with random packing. In a particularly preferred embodiment, the extraction is carried out in a column with structured packing.

In the extraction, the phase ratio of polar to organic phase is in a range from 0.1 to 2. Preference is given to a phase ratio in the range from 0.15 to 1.5, more preferably from 0.2 to 1 and in particular from 0.3 to 0.5.

When a column with structured packing is used for extraction, in a preferred embodiment, the polar phase is preferably drawn off as the extract via the bottom of the column; the raffinate, the organic phase, preferably runs off as a free overflow. The extraneous phase content, i.e. the alkanol, in the extract is preferably removed by a drawn-loop plastics knit in the bottom of the column.

When water is used as the polar extractant, the extraneous phase content, i.e. the content of undissolved water, in the raffinate after the phase separation is generally about 1 percent.

The raffinate comprises the desired product. Should the content of polar and ionic components in the raffinate be greater than the required product specification allows, it is possible in a preferred embodiment to recycle the raffinate into the feed. In this case, the raffinate may, for example, either be fed directly into the feed vessel for extraction or into a buffer vessel from which the extraction is fed.

The polar and ionic components are, for example, alkali metal or alkaline earth metal salts, alkali metal or alkaline earth metal alkoxides, acid, cyclic or acyclic mono- and/or diesters of carbonic acid, alkan(edi)ols and also polar melamine derivatives. Alkanediols are, for example glycol and propanediol; polar melamine derivatives are, for example, melamine, mono- and dialkoxycarbonylaminotriazines.

The possibility of removing mono- and dialkoxycarbonylaminotriazines from the raffinate by the extraction makes it possible to adjust, in a controlled manner, the ratios of different alkoxycarbonylaminotriazines in the raffinate.

The extraction in the column with structured packing is carried out generally as a countercurrent extraction. In a particularly preferred embodiment, this is done by feeding the polar extractant above the packing and the alkanolic reaction mixture below the packing. Within the column, the polar extractant thus flows through the packing in the direction of the column bottom and the alkanolic reaction mixture through the packing in the direction of the column top. The alkanolic reaction mixture and the polar extractant mix in the packing, and the ionic and/or polar components present in the alkanolic reaction mixture are passed to the polar extractant and thus removed from the alkanolic reaction mixture.

The temperature at which the extraction is carried out is preferably in the range from 10 to 90° C., more preferably in the range from 15 to 50° C.

A preferred pressure at which the extraction is carried out is ambient pressure. However, it is also possible to carry out the extraction at a pressure below ambient pressure or else at an elevated pressure. When the extraction is carried out at elevated pressure, the pressure is preferably in the range from 1 to 10 bar.

When the pH of the reaction mixture deviates from a desired pH, it can be adjusted by adding an acid or a base. The pH of the reaction mixture should generally be between 2 and 8, preferably between 2 and 7, and more preferably between 4.2 and 6.5. The pH can be adjusted either before the extraction or during the extraction.

In the case of too low a pH, it can be increased by adding a base. All bases are suitable for this purpose. Preference is given to using NaOH or aqueous ammonia (NH₄OH in aqueous solution). Particular preference is given to aqueous ammonia.

Too high a pH can be lowered to the desired value by adding an acid. Suitable acids for this purpose are both mineral and organic acids. Preference is given to organic acids and particular preference to formic acid.

In one embodiment, ionic components present in the reaction mixture may additionally be removed by an ion exchange over a cation exchanger and/or anion exchanger. The ion exchange may be carried out either before the extraction or after it.

The cation exchanger and/or the anion exchanger may be present, for example, as fixed bed ion exchanger(s). It is also possible for the cation exchanger and/or the anion exchanger to be present as granule. The cation exchanger and/or the anion exchanger may be present in a vessel, for example a stirred tank, a column or in another apparatus known to those skilled in the art. The cation exchanger and/or anion exchanger are preferably present in one column.

In a further embodiment, it is also possible to feed the cation exchanger and/or anion exchanger as granule to the reactor in which reactor the alkoxycarbonylaminotriazine is prepared. The reactor in this case is preferably a stirred tank.

In a preferred embodiment, the alkali metal and/or alkaline earth metal ions are removed from the alkanotic reaction mixture with a cation exchanger.

It is also possible to remove anions with an anion exchanger. For example, the reaction mixture may comprise nitrate, sulfate or phosphate ions, or else the anions or organic acids such as formic acid as a result of the neutralization of the reaction mixture with an acid, and are removed by the ion exchange with the anion exchanger.

Regeneration of the laden anion exchanger is effected preferably with dilute mineral alkalis. Particularly suitable for regeneration of the anion exchanger is 5-25% sodium hydroxide solution.

The cation exchanger is regenerated preferably with dilute mineral acids. A suitable mineral acid is, for example, 5-30% hydrochloric acid.

To pass through several cycles, both the anion exchange resin and the cation exchange resin are generally pretreated with a solubilizer between organic and polar phase. To this end, the ion exchanger is rinsed with a substance which has a polarity between the polarity of the organic phase and of the polar phase and is preferably miscible with both phases. For example, methanol is suitable as a solubilizer when butanol is the organic phase and water is the polar phase. In addition to a regeneration of the ion exchange resin, it is also conceivable to discard the laden resin without regeneration.

Anion exchangers suitable in accordance with the invention are, for example, strongly basic anion exchange resins. Preference is given to crosslinked polystyrene resins or styrene-divinylbenzene copolymers with tertiary or quaternary amines as functional groups and OH⁻ ions as exchange ions. Exchange ions are understood to mean the ions which are bonded to the functional groups and are exchanged for the ions to be removed from the liquid. In commercially available anion exchangers, the functional groups are generally present as salts. In these anion exchangers, CI⁻ ions, for example, are bonded to the functional group. In order to be able to use the anion exchangers, it is in this case generally first pretreated with NaOH in order to exchange the CI⁻ ions for OH⁻ ions. Suitable commercially available anion exchangers are, for example, Lewatit® MP62, Lewatit® 64 or Lewatit® MP 600 WS from Bayer AG, or else Amberjet® 4200 CL or Ambersep® 900 OH from Rohm & Haas Co. For the removal of nitrate ions, preference is given to Ambersep® 900 OH and Lewatit® M 600 WS, particular preference to Ambersep® 900 OH.

Suitable cation exchangers are, for example, strongly acidic cation exchanger resins based on a crosslinked polystyrene matrix or a styrene-divinylbenzene copolymer matrix and sulfonic acid as the functional group with H⁺ ions as exchange ions. In general, the cation exchangers, just like the anion exchangers, are in their salt form when available commercially. In order to be able to use the cation exchanger, it is then generally pretreated with an acid, for example sulfuric acid, in order to exchange the cations of the salt for H⁺ ions. Commercially available, suitable cation exchangers are, for example, Lewatit® S2528 or Lewatit® MonoPlus® S100 from Bayer AG, Amberlyst® 40 WET and Amberjet® 1500 H from Rohm & Haas Co., and also Dowex® N306 from Dow Chemical Co. for the removal of sodium ions, for example, preference is given to using Amberlyst® 40 WET and Amberjet® 1500 H.

The cation exchanger and the anion exchanger may be used either together as a mixture, individually or in steps or stages connected in series. Suitable combinations of suitable commercially available anion exchangers and cation exchangers are, for the removal of nitrate salts, Ambersep® 900 OH or Amberjet® 4200 as the anion exchanger and Lewatit® S2528 as the cation exchanger. Preference is given to the combination of Ambersep® 900 OH and Lewatit® S2528.

The alkanolic reaction mixture can be contacted with the cation exchanger and/or anion exchanger, for example, by adding the cation exchanger and/or anion exchanger to the reaction mixture, for example into the reactor or into a stirred vessel, or by flowing the reaction mixture through a continuous ion exchanger, in which case the ion exchanger is present, for example, as a packing in a fixed bed.

The addition of the ion exchange resin into the reaction vessel is possible especially when the at least one alkoxycarbonylaminotriazine is prepared batchwise. In this case, preference is given to effecting both the preparation of the at least one alkoxycarbonylaminotriazine and, if appropriate, a neutralization of the reaction mixture and the removal of the ionic components by ion exchange in the same vessel.

In one embodiment the process may comprise, as an additional step, the concentration of the organic phase comprising at least one alkoxycarbonylaminotriazine.

The concentration can be effected by thermal or mechanical processes. Suitable thermal processes for the concentration are, for example, evaporation, distillation, rectification, drying, preferably spray drying or crystallization. Suitable mechanical processes are in particular membrane separation processes, for example pervaporation or permeation, and also filtration when the at least one alkoxycarbonylaminotriazine is present as a suspension. The processes for concentration may each be employed individually or in combination. It is also possible to use any further process for concentration known to those skilled in the art. Preferred processes for concentration are distillation and spray drying.

In the case of concentration by distillation, it can be carried out before or after the extraction. When an ion exchange is carried out additionally, the distillation can also be carried out before or after the ion exchange, preference being given to carrying out the distillation before the ion exchange, since distillation reduces the volume stream comprising the alkoxycarbonylaminotriazine and thus smaller apparatus can be used to carry out the ion exchange. This lowers both the capital and the operating costs for the ion exchange.

The concentration of the organic phase comprising alkoxycarbonylaminotriazine by distillation can be effected continuously or batchwise.

For the continuous distillation, it is possible to use conventional continuous evaporators known to those skilled in the art. Suitable evaporators for continuous distillation are, for example, circulation evaporators such as Robert self-circulation evaporators, rapid-circulation evaporators with inclined evaporator tubes, forced-circulation evaporators with external evaporator bundles, circulation evaporators with boiling space divided into chambers, or forced-circulation evaporators with horizontal heater. Further suitable continuous evaporators are, for example, falling-film evaporators, thin-film evaporators or Kästner evaporators.

In addition, the organic phase can be concentrated by distillation in a column. The heating to evaporation temperature can be effected at the bottom of the column or in a heat exchanger disposed outside the column. Suitable columns are, for example, columns with structured packing or random packing, or tray columns. Suitable structured packings, random packings or trays are all structured packings, random packings or trays known to those skilled in the art.

Batchwise concentration by distillation can be effected, for example, in a stirred vessel. The distillation can also be carried out in the vessel in which the reaction to give alkoxycarbonylaminotriazine is carried out. Preference is given to effecting the concentration by distillation in an additional stirred vessel.

Both in the continuous and in the batchwise process, the product stream comprising alkoxycarbonylaminotriazine is obtained as the liquid phase and at least one vapor stream comprising alkanol, carbonate and water. When a polar extractant different from water is used in the removal by extraction of salts formed by the neutralization, the polar extractant is present in the vapor either instead of the water or in addition.

The concentration of the organic phase comprising alkoxycarbonylaminotriazine by distillation leads, in a particularly preferred embodiment, to a product stream which comprises 45-60% by weight of alkoxycarbonylaminotriazine.

However, depending on the desired product stream, it is also possible for the distillation to afford a product stream which comprises a relatively small or even a relatively large proportion of alkoxycarbonylaminotriazine.

In a further process variant, in the concentration of the organic phase comprising at least one alkoxycarbonylaminotriazine, the organic phase and the polar phase are removed by distillation. To this end, the organic phase comprising at least one alkoxycarbonylaminotriazone is fed to a distillation column. In this case, the distillation column preferably comprises a rectifying section and a stripping section. The organic phase comprising at least one alkoxycarbonylaminotriazine is fed preferably via a side feed in the rectifying section.

At the bottom of the distillation column, the concentrated organic phase comprising alkoxycarbonylaminotriazine is obtained.

In a preferred embodiment, the thus obtained bottom product is utilized in a heat exchanger to heat the desalted organic phase which is fed as feed to the distillation column.

Via the top of the distillation column, alkanols, if appropriate low boilers and water and/or polar extractant are drawn off. In a particularly preferred embodiment, this stream is subsequently fed to a phase separator in which the polar phase is separated from the organic phase. The organic phase is preferably fed as reflux back to the distillation column at the top thereof.

In a particularly preferred embodiment, the distillation column comprises a side draw arranged preferably in the stripping section, via which a preferably vaporous and substantially anhydrous stream comprising carbonate and alcohol is drawn off A particularly preferred position of the side draw is directly above the column bottom or directly below the separating internals in the column.

Advantages of this operating mode lie in the recovery of a majority of the carbonates via side draw for reuse in the reaction, the reduction in the carbonate content in the product stream, and in the recovery of anhydrous high-boiling alkanols, for example n-butanol, via the side draw.

In a preferred embodiment, the distillation column comprises from 8 to 22 theoretical plates.

The reflux ratio of the organic phase at the top of the column is preferably in the range between 0.2 and 3 kg/kg.

The distillation column is preferably operated with a pressure in the range between 20 and 2000 mbar at the top of the column. The preferred range in which the column is operated is between 50 and 950 mbar.

In a further process variant it is possible, in addition to the concentration of the phase comprising alkoxycarbonylaminotriazine by distillation or instead of the concentration by distillation, to provide a spray drying as a further process step. The spray drying generates pulverulent alkoxycarbonylaminotriazine.

Preference is given to effecting the spray drying in a spray drier as is known to those skilled in the aft. For example, commercial spray driers with atomizer disk, one-substance nozzle or two-substance nozzle may be used for the spray drying. Depending on the design, operation can be effected in cocurrent or in countercurrent. Preference is given to using a two-substance nozzle in which the liquid phase comprising at least one alkoxycarbonylaminotriazine is atomized at ambient pressure with the aid of a nitrogen stream. The nitrogen stream has a pressure in the range from 1 to 10 bar, preferably in the range from 2 to 5 bar and more preferably in the range from 2.5 to 5 bar. The nitrogen is used preferably as the cycle gas.

The spray drying is carried out preferably at a temperature in the range from 50 to 250° C., preferably at a temperature of from 55 to 150° C. and more preferably at a temperature in the range from 6 to 100® C., and at ambient pressure or an elevated pressure or reduced pressure of up to +/−0.01 MPa based on ambient pressure.

The pulverulent product generated in the spray drying may be removed, for example, in a fabric filter of customary design, such as candle filters, bag filters, hose filters or other filters known to those skilled in the art, or in a cyclone. Suitable filter material for a fabric filter is, for example, polytetrafluoroethylene, silicone or polyester. Preference is given to polyester.

The nitrogen used as the cycle gas is purified preferably in a scrubber. It is possible to use any scrubber known to those skilled in the art.

### EXAMPLES

### Example 1

A reaction mixture which has been prepared using dimethyl carbonate and comprises alkoxycarbonylaminotriazine is worked up after the neutralization has been carried out with 30% HNO₃. To this end, 63.6 kg/h of the organic homogeneous phase removed in a phase separator with a water content of 13%, an Na content of 3120 mg/kg and an NO₃⁻ content of 7440 mg/kg are washed in countercurrent with 22.3 kg/h of fully demineralized water in a column with structured packing. The liquid hourly space velocity on the column is 19.2 m³/m²h, which corresponds to 75% of the flood point value. The temperature at which washing is effected is 21° C. This gives rise to a raffinate stream of 60.7 kg/h and an extract stream of 28.2 kg/h. The raffinate stream constitutes the product of value comprising alkoxycarbonylaminotriazine. The analysis of the sodium ions and nitrate ions of the washed organic phase gives rise to a concentration of 1 mg/kg Na⁺ and 48 mg/kg NO₃⁻. The water content of the organic phase is 19.7%.

### Example 2

31.9 kg/h of the organic phase comprising alkoxycarbonylaminotriazine are fed to the column with structured packing after neutralization with 30% HNO₃. The organic phase is washed in the column with 12.5 kg/h of fully demineralized water in countercurrent. The liquid hourly space velocity on the column is 9.9 M³/m²h, which corresponds to 37% of the flood point value. From the column with structured packing, a raffinate stream comprising alkoxycarbonylaminotriazine of 28.2 kg/h and an extract stream of 13.9 kg/h are drawn off. The water content of the washed organic phase is 19.0%. The concentration of sodium ions is 1 mg/kg, the concentration of nitrate ions 36 mg/kg.

### Example 3

A reaction mixture prepared using ethylene carbonate is worked up after neutralization with 30% HNO₃. To this end, 17.10 kg/h of the organic, homogeneous phase comprising alkoxycarbonylaminotriazine which has been drawn off and has a water content of 10%, an Na content of 2640 mg/kg and an NO₃⁻ content of 7320 mg/kg are washed in a column with structured packing with 6.0 kg/h of water in countercurrent. The liquid hourly space velocity on the column is 20.6 m³/m²h, which corresponds to 59% of the flood point value. The temperature at which the washing is carried out is 21° C. This gives rise to a raffinate stream comprising alkoxycarbonylaminotriazine of 16.7 kg/h and an extract stream of 6.5 kg/h. The raffinate stream comprises 2 mg/kg of sodium ions and 12 mg/kg of nitrate ions. The water content of the raffinate stream is 16.5%.

1-17. (canceled)

18. A process comprising:
providing an alkanolic reaction mixture, the alkanolic reaction mixture comprising: at least one alkoxycarbonylaminotriazine; at least one carbonic ester; at least one C₁-C₁₃-alkanol, which alkanol may further comprise up to two ether-bound oxygen atoms and may further comprise a substituent selected from the group consisting of C₁-C₄-alkyls and hydroxyls; and at least one alkali metal alkoxide or alkaline earth metal alkoxide; and
subjecting the alkanolic reaction mixture to extraction with a polar extractant such that at least a portion of polar/ionic components present in the reaction mixture are removed, wherein the polar extractant is less than entirely miscible with an organic phase of the reaction mixture, to form an alkanolic phase comprising the at least one alkoxycarbonylaminotriazine and a polar phase comprising the polar extractant and the portion of polar/ionic components; and wherein the extraction is carried out with one or more selected from the group consisting of mixer/settler units, columns, centrifugal field separation extractors, and combinations thereof.

19. The process according to claim 18, wherein the extraction is carried out with a column having at least one selected from the group consisting of random packing, structured packing, and trays.

20. The process according to claim 18, wherein the extraction is carried out with a stirred or pulsed column.

21. The process according to claim 18, wherein the extraction is carried out at a temperature of 20 to 80° C.

22. The process according to claim 18, wherein the polar phase and the organic phase are present in a phase ratio of 0.1 to 2.

23. The process according to claim 18, wherein the polar extractant comprises water.

24. The process according to claim 18, further comprising concentrating the organic phase comprising at least one alkoxycarbonylaminotriazine.

25. The process according to claim 18, wherein the the alkali metal alkoxide comprises sodium methoxide.

26. The process according to claim 18, wherein the C₁-C₁₃-alkanol comprises butanol.

27. The process according to claim 18, wherein the C₁-C₁₃-alkanol comprises a mixture of methanol and butanol.

28. The process according to claim 18, further comprising neutralizing the alkanolic reaction mixture with an acid prior to extraction.

29. The process according to claim 28, wherein the acid comprises nitric acid.

30. The process according to claim 18, further comprising adjusting the pH of the alkanolic reaction mixture.

31. The process according to claim 30, wherein adjusting the pH of the alkanolic reaction mixture comprises adding aqueous ammonia to the reaction mixture.

32. The process according to claim 30, wherein adjusting the pH of the alkanolic reaction mixture comprises adding an organic acid.

33. The process according to claim 30, wherein adjusting the pH of the alkanolic reaction mixture comprises adding formic acid.

34. The process according to claim 18, further comprising spray-drying the organic phase to form a powder comprising the at least one alkoxycarbonyl-aminotriazine.

35. The process according to claim 24, further comprising spray-drying the organic phase to form a powder comprising the at least one alkoxycarbonyl-aminotriazine.

36. The process according to claim 34, wherein the spray-drying is carried out at a temperature of 50 to 250° C.

37. The process according to claim 35, wherein the spray-drying is carried out at a temperature of 50 to 250° C.
